(19) United States
(12) Patent Application Publication
(10) Pub. No.: US 2007/0112234 A1
(43) Pub. Date: May 17, 2007

(54) METHOD AND APPARATUS FOR PREPARING VINYL CHLORIDE USING ETHANE AND 1,2-DICHLOROETHANE

(75) Inventors: Sung Won KIM, Daejeon (KR); DongHyun JO, Daejeon-city (KR); Ye Hoon KIM, Daejeon-city (KR); Juyoul KIM, Hwaseong-gun (KR); Soonyeel LEE, Daejeon-city (KR); Byungchul OH, Daejeon-city (KR); Seung Back HA, Hanam-city (KR); Gimoon NAM, Seoul (KR); Ju Young YOUN, Seoul (KR); Sang-Seung OH, Cheonan-city (KR); Jongwook BAE, Daejeon-city (KR)

Correspondence Address: CANTOR COLBURN, LLP, 55 GRIFFIN ROAD SOUTH, BLOOMFIELD, CT, 06002, US

(73) Assignee: LG CHEM, LTD., Seoul (KR)

(21) Appl. No.: 11/560,069

(22) Filed: Nov. 15, 2006

(30) Foreign Application Priority Data
Nov. 15, 2005 (KR) 10-2005-0108941
Sep. 8, 2006 (KR) 10-2006-0086997

(51) Int. Cl. C07C 17/10

(52) U.S. Cl. 570/230

(57) ABSTRACT

The present invention provides a method and apparatus for preparing vinyl chloride in which reaction yield is improved and problems caused by coke generated during reactions can be solved. According to an aspect of the present invention, there is provided a method of preparing vinyl chloride comprising: supplying chlorine gas and ethane to an ethane chlorination reaction region disposed in a lower portion of a pyrolysis reactor in which solid particles exist; performing an ethane chlorination reaction by contacting the chlorine gas and ethane with solid particles such that a product of the ethane chlorination reaction and the solid particles rise toward an upper portion of the pyrolysis reactor at the same time, and depositing coke produced during the ethane chlorination reaction on the solid particles; performing a pyrolysis reaction in a pyrolysis reaction region disposed in an upper portion of the pyrolysis reactor by contacting a product of the ethane chlorination reaction with the solid particles such that the product of the ethane chlorination reaction and the solid particles rise up at the same time, and depositing coke produced during the pyrolysis reaction on the solid particles; separating solid particles obtained from the pyrolysis reaction and a product of the pyrolysis reaction in a separator; moving the separated solid particles to a regeneration reactor, and then burning coke deposited on the solid particles to regenerate the solid particles; and resupplying the regenerated solid particles to the pyrolysis reactor. According to another aspect of the present invention, there is provided an apparatus of preparing vinyl chloride comprising: a pyrolysis reactor comprising an ethane chlorination reaction region in a lower portion thereof and a pyrolysis reaction region in an upper portion thereof; a separator that separates a product of a pyrolysis reaction and solid particles; and a regeneration reactor that regenerates the separated solid particles by burning. When vinyl chloride is prepared using the method and apparatus according to the present invention, reaction yield is improved, and coke production and following coke accumulation in reactor can be inhibited.

### CROSS-REFERENCE TO RELATED PATENT APPLICATION

This application claims the benefit of Korean Patent Application No. 10-2005-0108941, filed on Nov. 15, 2005 and No. 10-2006-0086997 filed on Sep. 08, 2006, in the Korean Intellectual Property Office, the disclosure of which is incorporated herein in its entirety by reference.

### BACKGROUND OF THE INVENTION

1. Field of the Invention

The present invention relates to a method and apparatus for preparing vinyl chloride using a chlorination reaction of ethane and a pyrolysis reaction of 1,2-dichloroethane, wherein two-step processes, reaction and reproduction, are possible. When vinyl chloride monomer is produced using the method according to the present invention, reaction yield is improved and problems caused by coke generated during the reactions can be solved.

2. Description of the Related Art

A method of preparing ethylene and vinyl chloride, involving using chlorine as a catalyst, by injecting ethane and chlorine into a high-temperature tubular reactor is disclosed in U.S. Pat. Nos. 5,097,083, 5,705,728 and the like.

However, the conventional method of preparing vinyl chloride requires additional processes such that ethylene produced with vinyl chloride through the pyrolysis reaction of ethane is separated, the separated ethylene is converted to 1,2-dichloroethane, and then pyrolysis reaction of 1,2-dichloroethane occurs again. Therefore, the conventional method of preparing vinyl chloride is complicated, and the cost of preparing vinyl chloride is increased according to ethylene yield. The method is widely used in industry for preparing vinyl chloride from ethylene, and the process is documented (Ulmann's Encyclopedia of Industrial Chemistry, 5^{th} Edition, 1986, vol.6, 287-289).

A pyrolysis reaction of ethane, using chlorine as a catalyst, is performed in a tubular reactor, and involves injecting ethane and chlorine to a tube, whereby the ethane and chlorine flow at a temperature of about 600-1,000° C. At this time, a severe exothermic reaction occurs while an initial ethane chlorination reaction is performed, and a large amount of coke is generated. Therefore, the coke is adhered to the inside of the tube, and an operation of the tubular reactor needs to be stopped regularly to remove the coke. In addition, to obtain a high yield, it is necessary that an inside temperature of the tubular reactor is controlled through efficient removal of the generated heat.

U.S. Pat. No. 5,705,728 discloses a method of improving a conversion rate and inhibiting coke production inside a reactor by turbulent mixing of two raw material gases such as ethylene and chlorine and adjustment of the molar ratio of the two gases in an entrance of the reactor. When this method is used, the amount of coke production can be reduced, but the generated coke cannot be removed during a reactor operation. In addition, the generated heat can be removed through only an outer wall of the reactor.

PCT Publication No. WO 95/26811 discloses a method and apparatus for efficiently performing a continuous exothermic reaction and endothermic reaction in a production of ethylene through an ethane chlorination reaction, wherein a reaction of ethane and chlorine, which is an exothermic reaction, forms ethyl chloride, and the produced ethyl chloride produces ethylene by an endothermic reaction in which pyrolysis occurs. In the method, an endothermic reaction is performed such that an inner tube is installed inside an outer tube, an exothermic reaction occurs in the inner tube, the generated heat is transferred to the outer tube, and then the heat is used. When the method is used, coke production can be relatively reduced, and the generated heat can be collected. However, stopping a reactor operation is inevitable for removing the generated coke, temperature within the tubes is locally raised due to irregularity in inside temperature distribution that is a characteristic of tubular reactors, and byproducts are increased. Therefore, improvements are required.

### SUMMARY OF THE INVENTION

The present invention provides a method and apparatus for preparing vinyl chloride in which reaction yield is improved and problems caused by coke generated during reactions can be solved.

According to an aspect of the present invention, there is provided a method of preparing vinyl chloride comprising: supplying a chlorine gas and ethane to an ethane chlorination reaction region disposed in a lower portion of a pyrolysis reactor in which solid particles exist; performing an ethane chlorination reaction by contacting the chlorine gas and ethane with solid particles to be gone up at the same time, and depositing coke produced during the reaction on the solid particles; performing a pyrolysis reaction in a pyrolysis reaction region disposed in an upper portion of the pyrolysis reactor by contacting a product of the ethane chlorination reaction with the solid particles to be gone up at the same time, and depositing coke produced during the reaction on the solid particles; separating solid particles obtained by the pyrolysis reaction and a product of the pyrolysis reaction in a separator; moving the separated solid particles to a regeneration reactor, and then burning coke deposited on the solid particles to regenerate the solid particles; and resupplying the regenerated solid particles to the pyrolysis reactor.

According to another aspect of the present invention, there is provided an apparatus of preparing vinyl chloride comprising: a pyrolysis reactor comprising an ethane chlorination reaction region in a lower portion and a pyrolysis reaction region in an upper portion; a separator that separates a product of pyrolysis reaction and solid particles; and a regeneration reactor that regenerates the separated solid particles by burning.

When vinyl chloride is prepared using the method and apparatus according to the present invention, reaction yield is improved, and coke production and following coke accumulation in reactor can be inhibited.

### BRIEF DESCRIPTION OF THE DRAWINGS

The above and other features and advantages of the present invention will become more apparent by describing in detail exemplary embodiments thereof with reference to the attached drawings in which:

FIGS. 1 through 3 are apparatuses for preparing vinyl chloride according to embodiments of the present invention.

### DETAILED DESCRIPTION OF THE INVENTION

Hereinafter, the present invention will be described in more detail by explaining embodiments of the invention with reference to the attached drawings.

To prepare vinyl chloride, an ethane chlorination reaction that chlorinates ethane using a chlorine gas as a catalyst and a pyrolysis reaction of 1,2-dichloroethane are performed. The reactions are consecutively performed in two different reaction regions of a reactor (pyrolysis reactor). The ethane chlorination reaction and the pyrolysis reaction are separately performed, and thus the two reactions can be performed under different conditions.

Here, ethane used as a reactant, and 1,2-dichloroethane, if necessary, are evaporated and then applied to the pyrolysis reactor. Besides the reactants, solid particles are added. Such solid particles cause the reactants be mixed while circulating in the pyrolysis reactor and a regeneration reactor, transfer reaction heat, and remove coke that is a byproduct.

First, the ethane chlorination reaction is performed such that immediately after chlorine gas and ethane supplied to a lower portion of the pyrolysis reactor contact the solid particles supplied from the regeneration reactor, the reactants rise toward an upper portion of the pyrolysis reactor. At this time, heat generated by an exothermic reaction, that is, the ethane chlorination reaction, is absorbed by the solid particles.

In the ethane chlorination reaction, which occurs in a lower portion of the pyrolysis reactor, chlorine gas and ethane are reacted at 400-800° C., and preferably 500-700° C., and at a pressure of 1-50 atm., and preferably 1-25 atm. When the ethane chlorination reaction is performed within such ranges of temperature and pressure, yield of a vinyl chloride monomer can be maximized, and coke production can be minimized.

Meanwhile, in order to minimize a cost of separating an unreacted product and coke production, a molar ratio of ethane and chlorine gas is 0.2-10, and preferably 0.5-5. In addition, in the region of the pyrolysis reactor where the ethane chlorination reaction occurs, a staying time of the reactants is maintained at 0.5-30 seconds, and preferably 1-15 seconds. Thus, byproducts resulting from the ethane chlorination reaction can be minimized.

The pyrolysis reaction, which occurs in an upper portion of the pyrolysis reactor is performed such that a product of the ethane chlorination reaction which rises from a lower portion of the pyrolysis reactor, and 1,2-dichloroethane injected from an upper portion of the ethane chlorination reaction region contact the solid particles, and rise up at the same time. At this time, the pyrolysis reaction is an endothermic reaction, and reaction heat required is provided by solid particles that absorb heat from the ethane chlorination reaction previously performed.

The pyrolysis reaction is performed at a temperature of 300-800° C., and preferably 400-700° C., and at a pressure of 1-50 atm, and preferably 1-25 atm. When the pyrolysis reaction is performed within such ranges of temperature and pressure, yield of vinyl chloride monomers can be maximized, and coke production can be minimized.

In addition, in the region of the pyrolysis reactor in which the pyrolysis reaction occurs, a staying time of the reactants including a product resulting from the region of ethane chlorination reaction is maintained at 0.05-20 seconds, and preferably 0.5-15 seconds. By doing this, byproduct production can be minimized. (When the pyrolysis reaction is performed in a fluidized bed reactor that uses a heating medium, coke deposited on a surface of the heating medium is reused through a reproduction process that uses oxygen.) When the ethane chlorination and pyrolysis reactions are performed in solids circulating fluidized bed reactor that uses a heating medium, coke deposited on a surface of the heating medium is reused as energy source through a regeneration process that uses oxygen for coke combustion.

According to an embodiment of the present invention, the ethane chlorination reaction is performed in a lower portion of the pyrolysis reactor, and the pyrolysis reaction is performed in an upper portion of the pyrolysis reactor. Therefore, a gas produced in the region of the pyrolysis reactor in which the ethane chlorination reaction occurs rises from a lower portion of the pyrolysis reactor with high temperature solid particles. Here, when 1,2-dichloroethane is injected, the produced gas and 1,2-dichloroethane are mixed by the solid particles, and thus pyrolysis reaction occurs.

That is, pyrolysis reaction of 1,2-dichloroethane, which is an endothermic reaction, is performed using heat generated by ethane chlorination reaction of ethane and chlorine gas, which is an exothermic reaction. Furthermore, the two reactions are performed in a single reactor, and thus reaction yield and reactor yield can be improved.

Gases in the pyrolysis reactor may be ethane and chlorine gas, which are reactants, and 1,2-dichloroethane, if necessary. However, non-active gases, such as nitrogen, argon, helium or the like, or constituents that do not interfere with pyrolysis reaction can be additionally included. It will be understood by those of ordinary skill in the art that adding constituents that are helpful for pyrolysis reaction may be made without departing from the spirit and scope of the present invention.

The pyrolysis reactor according to the current embodiment of the present invention can be a tubular reactor, a fluidized bed reactor that uses a heating medium or the like, and preferably a fluidized bed reactor.

In the current embodiment of the present invention, by applying a fluidization or fluidized bed technique, and particularly a circulating fluidizing bed technique in which ethane chlorination and pyrolysis are performed by solid particle flow, high yield can be obtained and also coke can be removed during operations.

A fluidization or fluidized bed technique is a technique that converts solid particles to have liquid-like characteristics by flowing a medium such as gas or liquid on a solid particle layer to float the solid particles, and is used in a process of treating solid particles or a pulverulent body. In addition, a circulating fluidized bed technique, which is a type of fluidized bed technique, is a technique in which a reaction occurs at a high gas flow velocity that can float and transfer all of a plurality of solid particles, and represents a high mixing efficiency and heat transfer efficiency. In such a circulating fluidized bed, the solid particles are transferred into a reactor, and then separated, and resupplied to the reactor through a recirculating unit. Therefore, from a perspective view of a whole system, particles are circulated to prepare a compound. When these techniques are used, performing two reactions in a single system is possible.

Therefore, in the current embodiment of the present invention, first, ethane and a chlorine gas are supplied to an apparatus in which high-temperature solid particles are circulated to perform the ethane chlorination reaction, which is an exothermic reaction. Then, the high-temperature solid particles that collect heat generated by the exothermic reaction are contacted with 1,2-dichloroethane supplied to an upper portion of the pyrolysis reactor to perform the pyrolysis reaction, which is an endothermic reaction, using the heat. As a result, the reaction is terminated. Another characteristic of the circulating fluidized bed in which solid particles within the pyrolysis reactor flow together with gases is that mixing of raw materials can be improved compared to when only raw material gases flow within the pyrolysis reactor. Thus, the number of initial sub-reactions can be reduced, and local hot spot of heat is reduced such that the solid particles absorb and transfer a large amount of heat generated in an initial conventional reaction.

The present invention provides a method of removing coke during operations that is a big cause of stopping a reactor operation. The method is performed such that coke, which is a reaction byproduct, is deposited on a surface of high-temperature solid particles to prevent carbon from being adhered to a reactor wall, the particles are released from the pyrolysis reactor and separated from the reaction gases, and then the particles are burned in a regeneration reactor in the presence of oxygen or air to remove coke. In addition, the solid particles are heated using heat generated during burning, and the heated solid particles are resupplied to the pyrolysis reactor, and thus the heat is used as a reaction heat.

Reaction conditions of solid particle regeneration due to coke burning are determined by a content of coke and a content of the solid particles. The reaction may be preferably performed at a temperature of 500-1,000° C., and more preferably at a temperature of 550-900° C.

The method of preparing vinyl chloride according to the current embodiment of the present invention is an auto-thermal reaction system that has a high conversion rate, and uses reaction heat obtained by burning coke, which is a reaction byproduct.

The solid particles according to the current embodiment of the present invention can be non-active solid particles such as alumina, silica, silica alumina or the like, or catalyst particles that help the ethane chlorination reaction or the pyrolysis reaction. Such particles may have a diameter of 5-1,000 μm, and preferably 10-300 μm.

The present invention also provides an apparatus for preparing vinyl chloride comprising: a pyrolysis reactor in which an ethane chlorination reaction occurs in a lower portion thereof and a pyrolysis reaction that occurs in an upper portion thereof; a separator that separates a product of pyrolysis reaction and solid particles; and a regeneration reactor that regenerates the separated solid particles by burning, wherein the apparatus is an apparatus in which particles are circulated.

The apparatus can further comprise a solid particle transferring unit interposed between the separator and the regeneration reactor, which does not contact gases generated from each of the separator and the regeneration reactor.

Meanwhile, the diameter of the pyrolysis reactor can be the same or different in the lower and upper portions of the pyrolysis reactor in which the ethane chlorination reaction and the pyrolysis reaction occur, respectively. When a staying time of the reactants is desired to be differently adjusted in each of the lower and upper portions of the pyrolysis reactor, the diameters of the lower and upper portions of the pyrolysis reactor are different. In the current embodiment of the present invention, the diameter of the lower portion of the pyrolysis reactor in which the ethane chlorination reaction occurs may be larger than the diameter of the upper portion of the pyrolysis reactor in which the pyrolysis reaction occurs.

The solid particles are released from the pyrolysis reactor with coke deposited thereon that is produced in the pyrolysis reactor. Then, these particles are separated from products of the pyrolysis reaction such as vinyl chloride, hydrogen chloride, ethylene, unreacted ethane, 1,2-dichloroethane and the like through cyclone, or a gas-solid separator that performs the same function. Thereafter, the solid particles are supplied to the regeneration reactor through a solid particle transferring unit that is designed in order for gases of the pyrolysis reactor and the regeneration reactor not to contact each other. Coke deposited on a surface of the solid particles can be removed by burning the solid particles supplied to the regeneration reactor in the presence of oxygen or air. Various types of regeneration reactors can be used as the regeneration reactor, and are not particularly limited. In the current embodiment of the present invention, a fluidized bed method in which the solid particles are burned while being floated may be used.

When a reaction temperature of the ethane chlorination reaction is different from a reaction temperature of the pyrolysis reaction, one or several solid particle transferring units can be further included between the regeneration reactor and the pyrolysis reactor. In addition, a heat exchanger is installed in each of the solid particle transferring units, and thus the solid particles can be supplied to the pyrolysis reactor by controlling the solid particles to have a desired temperature.

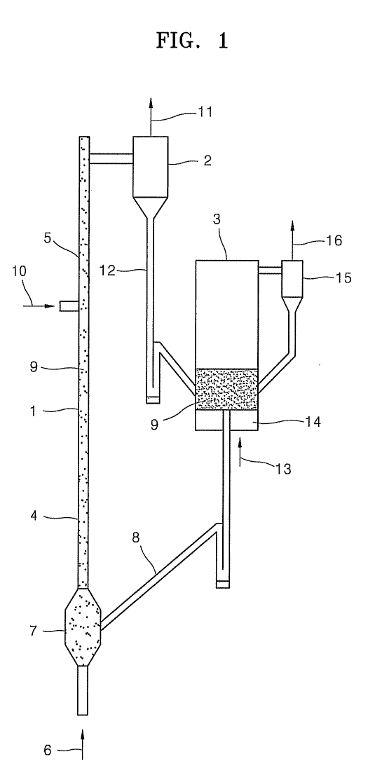

FIGS. 1 through 3 are apparatuses for preparing vinyl chloride according to embodiments of the present invention. Referring to FIG. 1, an apparatus for preparing vinyl chloride according to an embodiment of the present invention comprises a pyrolysis reactor 1, a gas-solid separator 2, which is a cyclone that separates solid particles from produced gases, and a regeneration reactor 3 that regenerates solid particles by burning coke deposited thereon. The pyrolysis reactor 1 largely comprises an ethane chlorination reaction region 4 and a pyrolysis reaction region 5. Ethane and chlorine gas 6 that are pre-heated with a desired temperature are supplied to a mixing chamber 7, and heated by heat generated by burning coke in the regeneration reactor 3 to be mixed with high-temperature solid particles 9 supplied through a solid particle transferring tube 8 that is a solid particle transferring unit. Here, a temperature of the ethane chlorination reaction region 4 is increased to a desired temperature in a lower portion of the pyrolysis reactor 1, and ethane and the chlorine gas 6 rise with the supplied solid particles 9 at a high speed to start an ethane chlorination reaction. A product of the ethane chlorination reaction is mixed with 1,2-dichloroethane 10 supplied from a desired position. At this time, pyrolysis reaction is performed in the pyrolysis reaction region 5 by the solid particles 9 that collect heat generated in the ethane chlorination reaction, while the reactants and 1,2-dichloroethane 10 rise together. Here, coke generated from the reaction is deposited on the solid particles 9, and released from the pyrolysis reactor 1 with the product. The coke and product are supplied into the gas-solid separator 2, which is a cyclone, in order to separate the gases and the solid particles 9. The generated gases and unreacted gas 11 are released from the pyrolysis reactor 1, and then cooled down and separated. The solid particles 9 on which coke is deposited are supplied to the regeneration reactor 3 through a solid particle transferring tube 12 that is connected to the regeneration reactor 3. At this time, in order for gases produced in the pyrolysis reactor 1 not to enter the regeneration reactor 3, nitrogen is injected to the particle transferring tube 12, and thus only the solid particles 9 enter the regeneration reactor 3. Air and methane 13 are injected to the regeneration reactor 3 through a distributing plate 14 for coke burning and raising temperature of the solid particles 9, and the temperature of the solid particles 9 is raised to a temperature required for coke burning and reaction through an operation of a fluidized bed. Some of the solid particles 9 are dispersed, thereby rising towards an upper portion of the regeneration reactor 3 with carbon dioxide and nitrogen generated by coke burning and methane burning. However, the solid particles 9 are collected by a cyclone 15 and then resupplied to the regeneration reactor 3, and produced combustion gases 16 are released outside of the regeneration reactor 3. The solid particles 9 raised to a desired temperature of the pyrolysis reactor 1 through combustion reaction are resupplied to the pyrolysis reactor 1 through a solid particle transferring tube 8, thereby being circulated and being used again in the pyrolysis reactor 1.

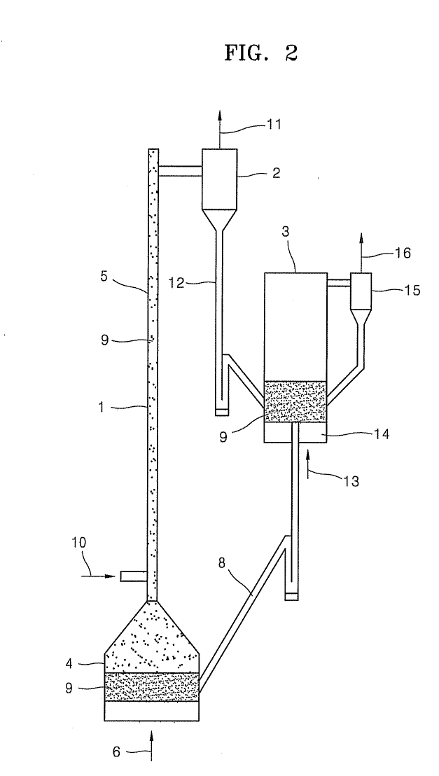

Meanwhile, the apparatus of FIG. 2 is the same as the apparatus of FIG. 1 in terms of a principle of solid particle flow and a whole reaction. However, the apparatus of FIG. 2 is different from the apparatus of FIG. 1 in order to take advantage of the fact that yield of vinyl chloride is high when a reaction time of an ethane chlorination reaction is relatively longer than a pyrolysis reaction. As heights of a reactor are changed, 1,2-dichloroethane can be injected in proportion to a length of a required staying time. However, there is possibility that a height of an apparatus is too high to design the apparatus, and a length of a solid particle transferring tube is also too long accordingly, and thus a flow of the solid particles is not smooth. Referring to FIG. 2, the diameter of an ethane chlorination reaction region 4 of a pyrolysis reactor 1 is larger than a diameter of a pyrolysis reaction region 5 of the pyrolysis reactor 1 in which 1,2-dichloroethane 10 is injected to and pyrolysis occurs. At this time, the diameter of the pyrolysis reactor 1 is proportionate to a staying time of the gas, and a staying time of solid particles 9 is also proportionate to the diameter pyrolysis reactor 1.

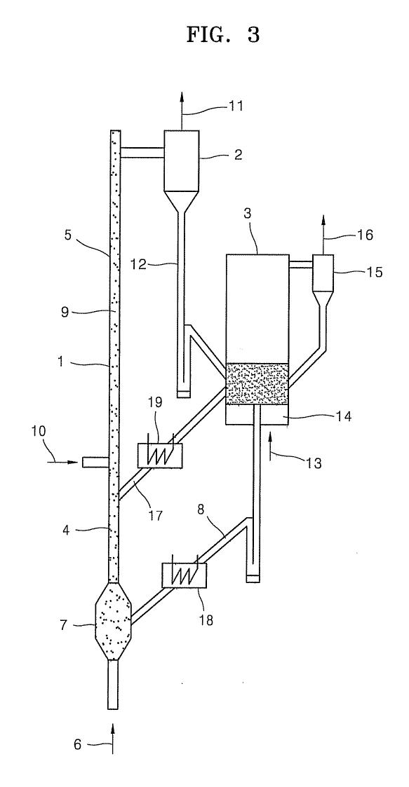

In addition, the apparatus of FIG. 3 is the same as the apparatus of FIG. 1 in terms of a solid particle flow and a whole reaction. However, the apparatus of FIG. 3 adopts the advantages that in terms of selecting temperature of an ethane chlorination reaction and a pyrolysis reaction, each region of the ethane chlorination reaction and the pyrolysis reaction can represent a maximum conversion rate and a maximum yield at a different temperature. In the apparatus of FIG. 3, two solid particle transferring tubes 8 and 17 are connected between a regeneration reactor 3 and a pyrolysis reactor 1. The solid particle transferring tube 8 is connected to an ethane chlorination reaction region 4, and the solid particle transferring tube 17 is connected to a pyrolysis reaction region 5 in which 1,2-dichloroethane 10 is injected to and thus pyrolysis occurs. In particular, heat exchangers 18 and 19 are installed on the solid particle transferring tubes 8 and 17 respectively, and thus solid particles 9 are injected to the pyrolysis reactor 1 by adjusting a temperature of the injected solid particles 9 to a desired temperature. When such a system is used, a desired temperature can be controlled and efficient collection of heat generated in the regeneration reactor 3 through coke combustion is also possible.

The embodiments of the present invention as illustrated in FIGS. 1 through 3 are for illustrative purposes only. The invention may, however, be embodied in many different forms, and any configuration of elements represented in the drawings do not depart from the spirit and scope of the present invention.

Hereinafter, the present invention will be described in further detail with reference to the following examples. These examples are for illustrative purposes only and are not intended to limit the scope of the present invention.

### EXAMPLE 1

(Reaction in a pyrolysis reactor)

A reaction was performed using an apparatus illustrated in FIG. 1. An Incolloy reactor in an ethane chlorination reaction region of a pyrolysis reactor having an external diameter of 1 inch and a length of 4 m was used. An Incolloy reactor in a pyrolysis reaction region of a pyrolysis reactor having an external diameter of 1 inch and a length of 3 m was used.

In the pyrolysis reactor, a pyrolysis reaction was performed by injecting ethane and chlorine gas to a lower portion of the pyrolysis reactor, and then injecting 1,2-dichloroethane at a position 4 m from the lower portion of the pyrolysis reactor.

A reaction of chlorine and ethane was performed in the ethane chlorination reaction region disposed in a lower portion of the pyrolysis reactor, and the reaction was performed at a chlorine gas/ethane molar ratio of 1.0, at a reaction temperature of 600° C., at a reaction pressure of 1.0 atm, and at a staying time of 6.0 seconds. Products in the ethane chlorination reaction region were all transferred into the pyrolysis reaction region. The pyrolysis reaction of the product of the ethane chlorination reaction and 1,2-dichloroethane that was additionally injected was performed in the pyrolysis reaction region disposed in an upper portion of the pyrolysis reactor. The pyrolysis reaction was performed at a reaction temperature of 500° C., and for 3.0 seconds including the product in the ethane chlorination reaction region.

The temperature of the pyrolysis reactor was adjusted by controlling an amount of high temperature alumina particles that were supplied from a regeneration reactor, and the flow rate (circulating amount) of the alumina particles was 25.8 g per second.

Raw material gases, ethane, chlorine and 1,2-dichloroethane, were injected into the pyrolysis reactor including the ethane chlorination reaction region and the pyrolysis reaction region at ratios of 42, 42 and 16 mole %, respectively.

A product released from the pyrolysis reactor was separated in a separator. The obtained elements are shown in Table 1 below.

TABLE 1

| pyrolysis reaction released element | Amount (mole %) |
|---|---|
| Ethylene | 3.57 |
| Ethane | 1.71 |
| Vinyl Chloride | 87.39 |
| Ethane chloride (C₂H₅Cl) | 0.11 |
| 1,2-dichloroethane | 1.26 |
| byproduct | 5.96 |

(Reaction in a Regeneration Reactor)

Coke deposited on the alumina particles was sent to a regeneration reactor with the alumina particles, and then burned. Reaction conditions of the regeneration reactor are shown in Table 2 below.

TABLE 2

| Reaction conditions | | Example 1 |
|---|---|---|
| regeneration | Circulating particle | alumina |
| reactor region | Particle circulating amount (g/s) | 25.8 |
| | Reaction temperature (° C.) | 740 |
| | Required air amount (g/min) | 73.62 |
| | Used methane amount (g/min) | 4.42 |

### EXAMPLE 2

(Reaction in a Pyrolysis Reactor)

A reaction was performed using an apparatus illustrated in FIG. 2. An Incolloy reactor in an ethane chlorination reaction region of a pyrolysis reactor having an external diameter of 1 inch and a length of 60 cm was used. The reaction was performed at a reaction temperature of 550° C., at a reaction pressure of 1.0 atm,at a staying time of 8 seconds, and at an ethane/chlorine molar ratio of 0.75. Products in the ethane chlorination reaction region were all transferred into the pyrolysis reaction region, and then a pyrolysis reaction was additionally performed with 1,2-dichloroethane. An Incolloy reactor in a pyrolysis reaction region of the pyrolysis reactor having an external diameter of ½ inches and a length of 90 cm was used. The pyrolysis reaction was performed at a reaction temperature of 500° C., and at a staying time of 2.5 seconds including a product in (a region A) the ethane chlorination region of the pyrolysis reactor.

The temperature of the pyrolysis reactor was adjusted by controlling an amount of high temperature alumina particles that were supplied from a regeneration reactor. The flow rate (circulating amount) of the alumina particles was 20.0 g per second.

Raw material gases, ethane, chlorine and 1,2-dichloroethane, were injected into the pyrolysis reactor including the ethane chlorination reaction region and the pyrolysis reaction region at ratios of 30, 40 and 30 mole %, respectively.

A product released from the pyrolysis reactor was separated in a separator. The obtained elements are shown in Table 3 below.

TABLE 3

| pyrolysis reaction released element | Example 2 |
|---|---|
| Ethylene | 3.97 |
| Ethane | 1.45 |
| Vinyl chloride | 79.55 |
| Ethane chloride (C₂H₅Cl) | 0.24 |
| 1,2-dichloroethane | 9.64 |
| byproduct | 5.15 |

Reaction in a Regeneration Reactor

Coke deposited on the alumina particles was sent to a regeneration reactor with the alumina particles, and then burned. Reaction conditions of the regeneration reactor are shown in Table 4 below.

TABLE 4

| Reaction conditions | | Example 2 |
|---|---|---|
| regeneration | Circulating particle | Alumina |
| reactor region | Particle circulating amount (g/s) | 20.0 |
| | Reaction temperature (° C.) | 780 |
| | Required air amount (g/min) | 57.36 |
| | Used methane amount (g/min) | 3.44 |

When a method of preparing vinyl chloride monomers according to the present invention is used, coke production can be inhibited, and high yield can be also achieved.

While the present invention has been particularly shown and described with reference to exemplary embodiments thereof, it will be understood by those of ordinary skill in the art that various changes in form and details may be: made therein without departing from the spirit and scope of the present invention as defined by the following claims.

What is claimed is:

1. A method of preparing vinyl chloride comprising:
supplying chlorine gas and ethane to an ethane chlorination reaction region disposed in a lower portion of a pyrolysis reactor in which solid particles exist;
performing an ethane chlorination reaction by contacting the chlorine gas and ethane with solid particles such that a product of the ethane chlorination reaction and the solid particles rise toward an upper portion of the pyrolysis reactor at the same time, and depositing coke produced during the ethane chlorination reaction on the solid particles;
performing a pyrolysis reaction in a pyrolysis reaction region disposed in an upper portion of the pyrolysis reactor by contacting a product of the ethane chlorination reaction with the solid particles such that the product of the ethane chlorination reaction and the solid particles rise up at the same time, and depositing coke produced during the pyrolysis reaction on the solid particles;
separating solid particles obtained from the pyrolysis reaction and a product of the pyrolysis reaction in a separator;
moving the separated solid particles to a regeneration reactor, and then burning coke deposited on the solid particles to regenerate the solid particles; and
resupplying the regenerated solid particles to the pyrolysis reactor.

2. The method of claim 1, further comprising supplying 1,2-dichloroethane to the pyrolysis reaction region disposed in an upper portion of the pyrolysis reactor, wherein the pyrolysis reaction is performed by contacting the additionally supplied 1,2-dichloroethane and the product of the ethane chlorination reaction with the solid particles such that the product of the ethane chlorination reaction and the solid particles rise up at the same time.

3. The method of claim 2, wherein 1,2-dichloroethane is vaporized, and then supplied to the pyrolysis reaction region disposed in an upper portion of the pyrolysis reactor.

4. The method of claim 1, wherein ethane is vaporized, and then supplied to the ethane chlorination reaction region disposed in a lower portion of the pyrolysis reactor.

5. The method of claim 1, wherein the pyrolysis reaction in the pyrolysis reaction region is performed partially or fully using heat energy of the solid particles that are high-temperature treated in the regeneration reactor.

6. The method of claim 1, wherein the solid particles are non-active solid particles selected from the group consisting of alumina, silica and silica alumina.

7. The method of claim 1, wherein the solid particles further comprise a catalyst particle in the ethane chlorination reaction or pyrolysis reaction.

8. The method of claim 1, wherein the solid particles have a diameter of 5-1,000 μm.

9. The method of claim 1, wherein the ethane chlorination reaction has a reaction temperature of 400-800° C.

10. The method of claim 1, wherein the ethane chlorination reaction has a reaction pressure of 1-25 atm.

11. The method of claim 1, wherein a molar ratio of ethane to chlorine gas in the ethane chlorination reaction is 0.5-5.

12. The method of claim 1, wherein the ethane chlorination reaction has a reaction time of 0.5-30 seconds.

13. The method of claim 1, wherein the pyrolysis reaction has a reaction temperature of 300-800° C.

14. The method of claim 1, wherein the pyrolysis reaction has a reaction pressure of 1-50 atm.

15. The method of claim 1, wherein the pyrolysis reaction has a reaction time of 0.05-20 seconds.

16. An apparatus for preparing vinyl chloride comprising:
a pyrolysis reactor comprising an ethane chlorination reaction region in a lower portion thereof and a pyrolysis reaction region in an upper portion thereof;
a separator that separates a product of a pyrolysis reaction and solid particles; and
a regeneration reactor that regenerates the separated solid particles by burning.

17. The apparatus of claim 16, wherein a solid particle transferring unit is interposed between the separator and the regeneration reactor not to contact a gas generated from the separator with a gas generated from the regeneration reactor.

18. The apparatus of claim 16, wherein at least one solid particle transferring unit is interposed between the regeneration reactor and the pyrolysis reactor.

19. The apparatus of claim 18, wherein a heat exchanger is installed in the at least one solid particle transferring unit in order to adjust the temperature of the solid particles.

20. The apparatus of claim 16, wherein the diameter of the ethane chlorination reaction region of the pyrolysis reactor is different from the diameter of the pyrolysis reaction region of the pyrolysis reactor.

21. The apparatus of claim 16, wherein the diameter of the ethane chlorination reaction region of the pyrolysis reactor is larger than the diameter of the pyrolysis reaction region of the pyrolysis reactor.
